(19) United States
(12) Patent Application Publication
(10) Pub. No.: US 2026/0286212 A1
(43) Pub. Date: Sep. 24, 2026

(54) CARBONATE REACTION PRODUCTS OF ALKYL GLYCOSIDES AND METHODS FOR USE THEREOF

(71) Applicant: SAUDI ARABIAN OIL COMPANY, Dhahran (SA)

(72) Inventors: Shaohua CHEN, Beijing (CN); Tianping HUANG, Beijing (CN); Ming HAN, Dhahran (SA)

(73) Assignee: SAUDI ARABIAN OIL COMPANY, Dhahran (SA)

(21) Appl. No.: 19/475,362

(22) PCT Filed: May 24, 2023
(86) PCT No.: PCT/CN2023/095941

§ 371 Date: Oct. 14, 2025

(51) Int. Cl. C09K 8/584; C09K 8/594; C09K 23/56

(52) U.S. Cl. CPC C09K 8/584; C09K 8/594; C09K 23/56

(57) ABSTRACT

Compositions comprising an organic carbonate salt having an anionic portion that is a carbonate reaction product of an alkyl glycoside may exhibit surfactant properties. When dissolved in an aqueous fluid, such organic carbonate salts may exhibit relatively low interfacial tension values and maintain tolerant to the presences of salts in the aqueous fluid. As a non-limiting example, aqueous fluids containing the organic carbonate salts may be introduced to a subterranean formation, such in conjunction with an enhanced oil recovery process.

### FIELD OF THE DISCLOSURE

The present disclosure generally relates to surfactants and, more specifically, surfactants exhibiting compatibility toward salt-containing environments and that are produced from a biologically sourced material.

### BACKGROUND OF THE DISCLOSURE

Compounds having both hydrophobic and hydrophilic regions within their molecular structure are amphiphilic and may exhibit surfactancy. As such, such compounds are commonly referred to as “surfactants” or “surfactant compounds.” By virtue of their molecular structure, surfactants tend to lower the interfacial tension at the interface between two substances. Because of their ability to lower interfacial tension, as well as facilitating other properties associated therewith (e.g., foaming, emulsification, de-emulsification, surface wetting, and the like), surfactants may be utilized in a wide range of consumer and industrial products and processes. Among such consumer and industrial products are, for example, soaps, detergents, cosmetics, pharmaceuticals, paints, and dispersants.

Surfactants are also commonly used in the oil and gas industry, such as during enhanced hydrocarbon recovery (e.g., enhanced oil recovery, EOR) processes. During EOR and similar downhole processes, surfactants may change wettability of a surface to promote hydrocarbon release therefrom, promote emulsificiation or de-emulsification of oleaginous substances, and/or reduce the viscosity of a fluid, as non-limiting examples.

Despite their wide-ranging use, there are difficulties associated with various conventional surfactants. A number of common surfactants exhibit poor biodegradability and/or varying degrees of aquatic toxicity. As such, these types of surfactants may be subject to various environmental and/or other government regulations. In addition, a number of surfactants are rather expensive and some may exhibit limited tolerance to environments having high salinity (e.g., about 5 wt. % salt or greater, including aqueous salt solutions, seawater, and brines). Finally, although surfactants may tend to lower interfacial tension at an interface between two substances, some aqueous surfactant solutions themselves may exhibit surface tension values that are higher than desired, especially at the critical micelle concentration. The limited salt tolerance may restrict the range of applications in which many conventional surfactants can be effectively used, and the high interfacial tension values may complicate fluid handling when using such surfactants.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

In various aspects, the present disclosure provides compositions comprising: an organic carbonate salt comprising an anionic portion that is a carbonate reaction product of an alkyl glycoside.

In other various aspects, the present disclosure provides methods for treating a subterranean formation, comprising: providing a composition comprising an organic carbonate salt comprising an anionic portion that is a carbonate reaction product of an alkyl glycoside; and introducing the composition into a subterranean formation.

In still other various aspects, the present disclosure provides methods for producing organic carbonate salts, comprising: contacting an alkyl glycoside with an elevated pressure of carbon dioxide in the presence of a non-nucleophilic base; and obtaining an organic carbonate salt comprising a cationic portion comprising the non-nucleophilic base in protonated form and an anionic portion that is a carbonate reaction product of the alkyl glycoside.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a plot of comparative FTIR spectra of an alkyl polyglycoside and an alkyl polyglycoside carbonate salt reaction product.

FIG. 2 is a plot of comparative UV-VIS spectra of an alkyl polyglycoside and an alkyl polyglycoside carbonate salt reaction product.

FIG. 3 is a plot of interfacial tension as a function of time for an alkyl glycoside and an alkyl glycoside carbonate salt reaction product.

### DETAILED DESCRIPTION

The present disclosure generally relates to surfactants and, more specifically, surfactants exhibiting compatibility toward salt-containing environments and that are produced from a biologically sourced material.

As discussed above, conventional surfactants may present various issues such as high cost, poor biocompatibility or biodegradability, limited compatibility with environments having high salinity, and high interfacial tension values. One or more of these factors may limit applicability of such surfactants in various applications. Surfactants that are incompatible with high-salinity environments may precipitate when contacted therewith, whereas the surfactant may remain soluble (compatible) when contacted with water or an aqueous fluid having sufficiently low salinity.

Alkyl glycosides are saccharide oligomers or polymers known to exhibit surfactancy and biodegradability. Alkyl glycosides comprise a saccharide polymer backbone having adjacent monosaccharide units linked by glycosidic bonds, wherein a terminal monosaccharide unit is functionalized with a long-chain alkyl group upon an oxygen atom that would otherwise form a glycosidic bond to another monosaccharide unit. Although alkyl glycosides may be useful non-ionic surfactants in various applications, alkyl glycosides may exhibit limited tolerance toward aqueous salt solutions. In addition, as-produced alkyl glycosides may demonstrate unacceptably high interfacial tension.

The present disclosure demonstrates that the properties of alkyl glycosides may be surprisingly enhanced by chemically transforming a parent alkyl glycoside from a neutral surfactant into an anionic surfactant. In particular, by functionalizing at least a portion of the monosaccharide units of the parent alkyl glycoside with an organic carbonate group, specifically as a salt of the organic carbonate group, increased tolerance toward aqueous salt solutions and similar high-salinity environments may be realized. Further surprisingly, following functionalization as a carbonate reaction product in salt form, decreased interfacial tension may be realized in comparison to the parent alkyl glycoside at a similar concentration in an aqueous fluid.

Advantageously, an organic carbonate group may be readily introduced to an alkyl glycoside and converted into a salt form using carbon dioxide in the presence of a suitably strong base, wherein the strong base may deprotonate the organic carbonate group to produce the salt form. Without being bound by theory or mechanism, the organic carbonate group is believed to form at the primary alcohol position of at least a portion of the monosaccharide units defining the saccharide polymer backbone (in the case of an alkyl glycoside comprising predominantly 1,4-glycosidic bonds). Any formation of organic carbonate groups at the secondary alcohol positions of the monosaccharide units within an alkyl glycoside comprising predominantly 1,4-glycosidic bonds is believed to be much more difficult due to increased steric hindrance. Further, organic carbonate groups introduced through a reaction with carbon dioxide in the presence of a suitably strong base may differ from cyclic carbonates formed by a reaction of adjacent hydroxyl groups with reagents such as phosgene.

Accordingly, compositions of the present disclosure may comprise an organic carbonate salt having an anionic portion that is a carbonate reaction product of an alkyl glycoside. Alkyl glycosides having all α(1,4) glycosidic bonds between adjacent glucose monomers may have a structure represented by Formula 1 below, wherein

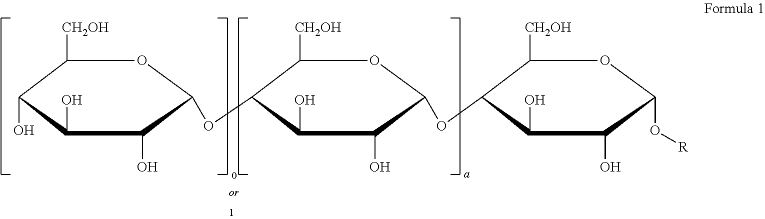

variable a is 0 or a positive integer, and R is an alkyl group upon the terminal glucose monomer unit, which defines the “alkyl” portion of the alkyl glycoside. Alkyl glycosides may have a molecular weight varying over a range of values, such as about 220 or above, or about 380 or above, or about 545 or above, or 700 or above. The alkyl group may comprise a C₄-C₂₄ alkyl group, or a C₆-C₂₀ alkyl group, or a C₈-C₁₈ alkyl group, or a C₆-C₁₄ alkyl group, or a C₁₀-C₁₆ alkyl group, or a C₁₂-C₁₄ alkyl group in non-limiting examples. It is to be appreciated that a given preparation of alkyl glycosides may contain a varying number of monosaccharide units, as well as a range of alkyl group sizes. Accordingly, in non-limiting examples, the molecular weight of suitable alkyl glycosides for use in the disclosure herein may range from about 220 to about 5000, or about 320 to about 2000, or about 320 to about 1000, or about 400 to about 800, or about 600 to about 1200, or about 350 to about 750. As few as one monosaccharide unit may be present, or as many as 2, 3, 4, 5, 10, 15, or even 20 monosaccharide units may be present in a parent alkyl glycoside prior to introduction of one or more organic carbonate groups thereto in salt form. Accordingly, it is to be appreciated that use of the term “alkyl glycoside” herein may refer to either alkyl monoglycosides (one monosaccharide unit) or alkyl polyglycosides (two or more monosaccharide units). It is to be further appreciated that alkyl glycosides having monosaccharide units other than glucose and/or glycosidic bonds other than α(1,4)-glycosidic bonds may also be suitable for use in the disclosure herein. For clarity, Formula 2 shows the IUPAC numbering of a single glucose monomer unit (in the form of an alkyl glycoside).

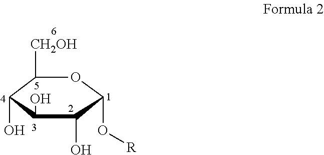

Organic carbonate salts of the present disclosure may contain at least one monosaccharide unit having a structure represented by Formula 3 below, wherein the C-1 and C-4 alcohol groups may be in the form of glycosidic bonds (in the case of alkyl polyglycosides), or the C-4 alcohol group of a terminal monosaccharide unit of an alkyl polyglycoside may be alkyl-functionalized.

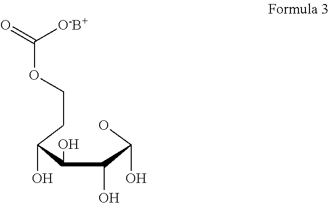

In Formula 3, B is the protonated form of a strong base.

The alkyl glycosides undergoing further functionalization to produce an organic carbonate salt according to the disclosure herein may be obtained commercially or synthesized in any suitable manner. In non-limiting examples, suitable alkyl glycosides may be synthesized by reacting a monosaccharide (e.g., glucose) or an oligosaccharide (e.g., multiple glucose monomer units linked by α(1,4)-glycosidic bonds) with a fatty alcohol or a mixture of fatty alcohols in the presence of a suitable acid catalyst. The acid catalyst may promote oligomerization (or further oligomerization) of the monosaccharide units, with the glycosidic bond position (i.e., the C-4 alcohol group in the case of glucose) of the terminal monosaccharide unit being etherified with the alkyl group of the fatty alcohol to form a plurality of alkyl glycosides. The plurality of alkyl glycosides may have a range of monosaccharide units linked by glycosidic bonds, each having the terminal monosaccharide unit functionalized with an alkyl group. A range of alkyl group sizes may be present when using a mixture of fatty alcohols. Alkyl glycosides formed through alternative syntheses may also be suitable for use in the disclosure herein.

As used herein, the term “fatty alcohol” refers to a long-chain primary alcohol compound bearing optional unsaturation and containing 4 or more carbon atoms, such as about 4 to about 26 carbon atoms, or about 6 to about 24 carbon atoms, or about 8 to about 20 carbon atoms, or about 10 to about 18 carbon atoms, or about 6 to about 14 carbon atoms, or about 10 to about 22 carbon atoms, or about 8 to about 16 carbon atoms. The fatty alcohol (or mixture of fatty alcohols) used to produce an alkyl glycoside may be formed via reduction of a fatty acid or fatty ester, preferably a straight-chain fatty acid or fatty ester. Accordingly, the alkyl glycosides disclosed herein may be free or substantially free of branched alkyl groups, according to various embodiments. Fatty acids may be directly reduced to the corresponding fatty alcohols using known techniques. Fatty esters (including fats or oils) may be directly reduced to the corresponding fatty alcohols using known techniques, or the fatty esters may first be hydrolyzed to fatty acids before undergoing reduction. Accordingly, the alkyl glycosides used herein may contain alkyl group(s) having a size commensurate with the fatty acid(s) or fatty ester(s) from which the fatty alcohols were produced.

Illustrative fatty acids (or fatty acids within fatty esters) from which the corresponding fatty alcohols may be obtained through various techniques include, for example, butyric acid, valeric acid, caproic acid, enanthic acid, caprylic acid, pelabonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, trioscylic acid, lignoceric acid, pentacosylic acid, cerotic acid, crotonic acid, cervonic acid, linoleic acid, linolelaidic acid, linolenic acid, arachidonic acid, docosatetraenoic acid, myristoleic acid, palmitoleic acid, sappenic acid, vaccenic acid, paullinic acid, oleic acid, pinolenic acid, stearidonic acid, eleostearic acid, elaidic acid, gondoic acid, gadoleic acid, erucic acid, eicosenoic acid, eicosadiencoic acid, eicosatrienoic acid, eicosatetraenoic acid, docosadienoic acid, nervonic acid, mead acid, adrenic acid, the like, and any combination thereof.

In non-limiting examples, glycerol esters may comprise a fatty ester source for obtaining fatty alcohols for forming an alkyl glycoside suitable for use herein. Suitable glycerol esters from which one or a mixture of fatty alcohols may be obtained through a suitable reduction technique include any plant oil, animal oil, plant fat, animal fat, or any combination thereof that contains one or more desired fatty acids. Suitable glycerol esters may be found in plant or animal sources including, for example, soybean oil, grapeseed oil, olive oil, palm oil, rice bran oil, safflower oil, corn oil, coconut oil, sunflower seed oil, canola oil, rapeseed oil, peanut oil, cottonseed oil, hazelnut oil, tea seed oil, linseed oil, sesame oil, acai oil, almond oil, beech nut oil, brazil nut oil, cashew oil, macadamia nut oil, pecan oil, pine nut oil, pistachio oil, walnut oil, pumpkin seed oil, apricot oil, avocado oil, grapefruit oil, lemon oil, orange oil, mango oil, flax seed oil, fish oil, cocoa butter, hemp oil, castor oil, tall oil, fish oil, cattle fat, buffalo fat, sheep fat, goat fat, duck fat, pig fat, poultry fat, and any combination thereof.

As noted above, formation of an organic carbonate salt may take place through a reaction with carbon dioxide in the presence of a suitable base. Suitable bases may include non-nucleophile bases, preferably a non-nucleophilic strong base having a pKₐ of about or greater. More preferably, the non-nucleophilic base may comprise an amidine, preferably a cyclic or bicyclic amidine. Examples of suitable amidines that may promote formation of an organic carbonate salt include, but are not limited to, 1,8-diazabicyclo[5.4.0]undec-7-ene (DBU), 1,5-diazabicyclo[4.3.0]non-5-ene (DBN), and any combination thereof. Accordingly, in some embodiments, a cation portion of the organic carbonate salts of the present disclosure may comprise DBU or DBN, specifically a protonated form of DBU or DBN.

Compositions of the present disclosure may be further formulated with an aqueous fluid in which the organic carbonate salt is at least partially dissolved and preferably fully dissolved. Aqueous fluids that may be present in the compositions disclosed herein include, but are not limited to, water, salt water, mixtures of water and a water-miscible organic solvent, brine, seawater, produced water, or any combination thereof. As used herein, the term “brine” refers to any aqueous salt solution having a greater amount of total dissolved salt than does seawater, including saturated aqueous salt solutions. Field or industrial process waters, including produced water from a wellbore, containing a high salt content may be suitably used. Compositions of the present disclosure may be formulated with an aqueous fluid that already has a high salt content (e.g., greater than 5 wt. % salt based on total mass of the aqueous fluid), while still remaining tolerant (stably formulated) in the presence of the high salt content. Alternately, the compositions of the present disclosure may be formulated with an aqueous fluid lacking a dissolved salt or an amount of dissolved salt that is not considered to constitute a high salt content. Moreover, the compositions may remain tolerant (stably formulated) when contacted with another aqueous fluid having a high salt content. Tolerance of the compositions toward high-salinity aqueous fluids may be evidenced by lack of precipitation or a decrease in clarity.

In the compositions of the present disclosure, the organic carbonate salt may be present in the aqueous fluid at a concentration of about 20 wt. % or less, or about 10 wt. % or less, or about 5 wt. % or less, such as about 1 wt. % to about 10 wt. %, or about 3 wt. % to about 8 wt. %, each based on total mass of the composition.

When contacted with an oleaginous substance, the compositions of the present disclosure may promote emulsification thereof, preferably as an oil-in-water emulsion. Advantageously, the emulsions may break spontaneously within about 24 hours, or within about 12 hours, or within about 8 hours, or within about 4 hours, or within about 2 hours, or within about 1 hour, or within about 30 minutes, or within about 20 minutes, or within about 10 minutes, or within about 5 minutes. Optionally, de-emulsification may be promoted by introducing a separate de-emulsifier.

Oleaginous substances that may be emulsified upon contacting the compositions of the present disclosure, including under high-salinity conditions, are not believed to be particularly limited. Illustrative oleaginous substances may include, for example, petroleum, refined petroleum, diesel, natural gas, plant oils, vegetable oils, an oilfield fluid or a component thereof, or any combination thereof.

Advantageously, the compositions of the present disclosure may exhibit low interfacial tension values. At the very least, the compositions of the present disclosure may exhibit a lower interfacial tension than does the corresponding alkyl glycoside prior to being converted into an organic carbonate salt. That is, when formulated in an aqueous fluid at a substantially identical concentration, the organic carbonate salts of an alkyl glycoside may exhibit a lower interfacial tension than does the corresponding unfunctionalized (parent) alkyl glycoside. In non-limiting examples, the interfacial tension of the organic carbonate salt may decrease by at least a factor of 5 relative to the unfunctionalized alkyl glycoside. More preferably, the interfacial tension may decrease by at least a factor of 10, or by at least a factor of 15, or by at least a factor of 20, or by at least a factor of 30, or by at least a factor of 40, or by at least a factor of 50 relative to the unfunctionalized alkyl glycoside. Alternately, at a concentration of 0.5 wt. % or less or 0.2 wt. % or less, interfacial tension values for the organic carbonate salts described herein may be about 0.3 mN/m or less, or about 0.25 mN/m or less, or about 0.2 mN/m or less, or about 0.15 mN/m or less, or about 0.1 mN/m or less, or about 0.05 mN/m or less, or about 0.01 mN/m or less.

As indicated above, the compositions of the present disclosure may be obtained by contacting an alkyl glycoside with an elevated pressure of carbon dioxide in the presence of a non-nucleophilic strong base (preferably an amidine, such as DBU or DBN), and obtaining an organic carbonate salt comprising a cationic portion comprising the non-nucleophilic base in protonated form and an anionic portion that is a carbonate reaction product of the alkyl glycoside.

Preferably, contacting the alkyl glycoside with the carbon dioxide may take place in an aprotic organic solvent (reaction solvent) under heating. In non-limiting examples, suitable aprotic organic solvents may include, for instance, dimethylformamide, dimethylsulfoxide, dimethylpropylene urea, the like, or any combination thereof. In some or other non-limiting examples, heating may take place at a temperature above room temperature (25° C.) up to about 100° C., or up to about 90° C., or up to about 80° C., up to about 70° C., or up to about 60° C. Suitable elevated pressures of carbon dioxide may range from about 2 atm to about 10 atm, or about 2.5 atm to about 7.5 atm, or about 3 atm to about 5 atm, or about 4 atm to about 6 atm.

After obtaining the organic carbonate salt upon contacting the alkyl glycoside with the carbon dioxide, methods of the present disclosure may further comprise isolating the organic carbonate salt from the reaction solvent. In non-limiting examples, the organic carbonate salt may be isolated using techniques such as, but not limited to, filtering, settling, decanting, centrifuging, or any combination thereof. After isolating the organic carbonate salt, the isolated organic carbonate salt may be further washed with a washing solvent to remove the reaction solvent. Suitable washing solvents may include those having a low boiling point and in which the organic carbon salt is insoluble but the reaction solvent is at least partially soluble. In non-limiting examples, suitable washing solvents may include, but are not limited to, hydrocarbon solvents such as hexane. Optionally, the organic carbonate salt may be further freeze dried or dried under vacuum and/or with heating to further remove the washing solvent, residual carbon dioxide, residual reaction solvent, or any combination thereof.

After obtaining the organic carbonate salt in accordance with the foregoing, the organic carbonate salt may be further dissolved in an aqueous fluid. Suitable aqueous fluids may include those specified above. A concentration of the organic carbonate salt in the aqueous fluid may include those specified above. In particular examples, the aqueous fluid may comprise at least one salt (e.g., brine, an aqueous salt solution, seawater, and the like).

The compositions of the present disclosure may be utilized in any application in which surfactancy is desired. In non-limiting examples, the compositions may be utilized in recovering hydrocarbons from a subterranean formation, such as in conjunction with an enhanced oil recovery process. In some or other non-limiting examples, the compositions may be introduced to a subterranean formation to promote emulsification of an oleaginous substance downhole or to promote de-emulsification of an existing emulsion downhole. Alternately, the compositions may be emulsified with an oleaginous substance prior to being introduced into a subterranean formation. In another example, the compositions may be utilized to change surface wetting characteristics (wettability) of a given surface downhole (e.g., a formation matrix or a fracture surface within a subterranean formation). Particular uses for the compositions and performance thereof may depend on the conditions present or anticipated to be present downhole.

In particular examples, compositions of the present disclosure may be used in conjunction with enhanced oil recovery (EOR) operations. When used in conjunction with EOR operations, the organic carbonate salts of the present disclosure may change surface wetting within a subterranean formation to promote recovery of hydrocarbons therefrom. For example, by changing the surface wetting characteristics of a subterranean formation, release of hydrocarbons from the formation matrix may take place more readily and/or the hydrocarbons may be mobilized toward a production well more readily. The foregoing may be facilitated through a variety of mechanisms including oil swelling, viscosity reduction, and wettability alteration, for example. The foregoing is typically referred to as the tertiary phase of hydrocarbon production, wherein one or more chemical or physical interactions are modified within the subterranean formation using an injected fluid to promote production. The tertiary phase of hydrocarbon production is usually employed after initial production resulting from native reservoir pressurization (primary production) or induced reservoir pressurization (secondary production) has been exhausted. Other details regarding enhanced oil recovery techniques will be familiar to one having ordinary skill in the art.

Embodiments disclosed herein include:
A. Compositions formed from alkyl glycosides. The compositions comprise: an organic carbonate salt comprising an anionic portion that is a carbonate reaction product of an alkyl glycoside.
B. Methods for treating a subterranean formation. The methods comprise: providing the composition of A; and introducing the composition into a subterranean formation.
C. Methods for forming an organic carbonate salt of an alkyl glycoside. The methods comprise: contacting an alkyl glycoside with an elevated pressure of carbon dioxide in the presence of a non-nucleophilic base; and obtaining an organic carbonate salt comprising a cationic portion comprising the non-nucleophilic base in protonated form and an anionic portion that is a carbonate reaction product of the alkyl glycoside.

Embodiments A, B, and C may comprise one or more of the following additional embodiments in any combination.

Element 1: wherein the alkyl glycoside is an alkyl polyglycoside.

Element 2: wherein the alkyl polyglycoside comprises at least two monosaccharide units, one or more of the at least two monosaccharide units are functionalized with an organic carbonate group in salt form, and a terminal monosaccharide unit of the at least two monosaccharide units is alkylated.

Element 3: wherein the at least two monosaccharide units are linked by α-1,4-glycosidic bonds.

Element 4: wherein the organic carbonate salt comprises a cation portion comprising a non-nucleophilic base in protonated form.

Element 5: wherein the non-nucleophilic base has a pKₐ of about 10 or greater.

Element 6: wherein the non-nucleophilic base comprises an amidine.

Element 7: wherein the cation portion comprises 1,8-diazabicyclo[5.4.0]undec-7-ene (DBU) in protonated form.

Element 8: wherein the alkyl glycoside comprises a terminal alkyl group comprising about 4 to about 24 carbon atoms.

Element 9: wherein the carbonate reaction product comprises an organic carbonate group in salt form at a primary alcohol position of at least one monosaccharide unit of the alkyl glycoside.

Element 10: wherein the composition further comprises an aqueous fluid in which the organic carbonate salt is dissolved.

Element 11: wherein the aqueous fluid comprises at least one salt.

Element 12: wherein the composition is introduced into the subterranean formation in conjunction with an enhanced hydrocarbon recovery process.

Element 13: wherein the composition contacts a salt-containing fluid in the subterranean formation, the aqueous fluid comprises at least one salt, or any combination thereof.

Element 14: wherein the method further comprises contacting the composition with an oleaginous substance in the subterranean formation; and emulsifying the oleaginous substance in the aqueous fluid in the presence of the organic carbonate salt.

Element 15: wherein the composition is emulsified with an oleaginous substance when introduced into the subterranean formation.

Element 16: wherein contacting takes place in an aprotic organic solvent under heating.

Element 17: wherein heating takes place at a temperature up to about 100° C.

Element 18: wherein the elevated pressure is about 3 atm to about 5 atm.

By way of non-limiting example, exemplary combinations applicable to A, B and C include, but are not limited to: 1 and 2, and 3; 1 and 2, and 4, 5, 6, or 7; 1 and 2, and 8; 1 and 2, and 9; 1 and 2, and 10; 1 and 2, and 10 and 11; 3, and 4, 5, 6, or 7; 3 and 8; 3 and 9; 3 and 10; 3, 10, and 11; 4, 5, 6, or 7, and 8; 4, 5, 6, or 7, and 9; 4, 5, 6, or 7, and 10; 4, 5, 6, or 7, and 10 and 11; 8 and 9; 8 and 10; 8, 10, and 11; 9 and 10; and 9-11. With respect to B and C any of the foregoing may be in further combination with one or more of 12-18. Further exemplary combinations applicable to B include, but are not limited to, 12 and 13; 12 and 14; 12 and 15; and 13 and 14. Further exemplary combinations applicable to C include, but are not limited to, 16 and 17; 16 and 18; 17 and 18; and 16-18.

The present disclosure is further directed to the following non-limiting clauses:
Clause 1. A composition comprising:
   an organic carbonate salt comprising an anionic portion that is a carbonate reaction product of an alkyl glycoside.
Clause 2. The composition of clause 1, wherein the alkyl glycoside is an alkyl polyglycoside.
Clause 3. The composition of clause 2, wherein the alkyl polyglycoside comprises at least two monosaccharide units, one or more of the at least two monosaccharide units are functionalized with an organic carbonate group in salt form, and a terminal monosaccharide unit of the at least two monosaccharide units is alkylated.
Clause 4. The composition of clause 3, wherein the at least two monosaccharide units are linked by α-1,4-glycosidic bonds.
Clause 5. The composition of any one of clauses 1-4, wherein the organic carbonate salt comprises a cation portion comprising a non-nucleophilic base in protonated form.
Clause 6. The composition of clause 5, wherein the non-nucleophilic base has a pKₐ of about 10 or greater.
Clause 7. The composition of clause 5 or clause 6, wherein the non-nucleophilic base comprises an amidine.
Clause 8. The composition of any one of clauses 5-7, wherein the cation portion comprises 1,8-diazabicyclo[5.4.0]undec-7-ene (DBU) in protonated form.
Clause 9. The composition of any one of clauses 1-8, wherein the alkyl glycoside comprises a terminal alkyl group comprising about 4 to about 24 carbon atoms.
Clause 10. The composition of any one of clauses 1-9, wherein the carbonate reaction product comprises an organic carbonate group in salt form at a primary alcohol position of at least one monosaccharide unit of the alkyl glycoside.
Clause 11. The composition of any one of clauses 1-10, further comprising:
   an aqueous fluid in which the organic carbonate salt is dissolved.
Clause 12. A method comprising:
   providing the composition of clause 11; and
   introducing the composition into a subterranean formation.
Clause 13. The method of clause 12, wherein the composition is introduced into the subterranean formation in conjunction with an enhanced hydrocarbon recovery process.
Clause 14. The method of clause 12 or clause 13, wherein the composition contacts a salt-containing fluid in the subterranean formation, the aqueous fluid comprises at least one salt, or any combination thereof.
Clause 15. The method of any one of clauses 12-14, further comprising:
   contacting the composition with an oleaginous substance in the subterranean formation; and emulsifying the oleaginous substance in the aqueous fluid in the presence of the organic carbonate salt.
Clause 16. The method of any one of clauses 12-14, wherein the composition is emulsified with an oleaginous substance when introduced into the subterranean formation.
Clause 17. A method comprising:
   contacting an alkyl glycoside with an elevated pressure of carbon dioxide in the presence of a non-nucleophilic base; and
   obtaining an organic carbonate salt comprising a cationic portion comprising the non-nucleophilic base in protonated form and an anionic portion that is a carbonate reaction product of the alkyl glycoside.
Clause 18. The method of clause 17, wherein contacting takes place in an aprotic organic solvent under heating.
Clause 19. The method of clause 18, wherein heating takes place at a temperature up to about 100° C.
Clause 20. The method of any one of clauses 17-19, wherein the elevated pressure is about 3 atm to about 5 atm.
Clause 21. The method of any one of clauses 17-20, wherein the alkyl glycoside is an alkyl polyglycoside.
Clause 22. The method of clause 21, wherein the alkyl polyglycoside comprises at least two monosaccharide units, one or more of the at least two monosaccharide units are functionalized with an organic carbonate group in salt form, and a terminal monosaccharide unit of the at least two monosaccharide units is alkylated.
Clause 23. The method of clause 22, wherein the at least two monosaccharide units are linked by α-1,4-glycosidic bonds.
Clause 24. The method of any one of clauses 17-23, wherein the non-nucleophilic base has a pKₐ of about 10 or greater.
Clause 25. The method of any one of clauses 17-24, wherein the non-nucleophilic base cation comprises an amidine.
Clause 26. The method of any one of clauses 17-25, wherein the cation portion comprises 1,8-diazabicyclo[5.4.0]undec-7-ene (DBU) in protonated form.
Clause 27. The method of any one of clauses 17-26, wherein the alkyl glycoside comprises a terminal alkyl group comprising about 4 to about 24 carbon atoms.
Clause 28. The method of any one of clauses 17-27, wherein the carbonate reaction product comprises an organic carbonate group in salt form at a primary alcohol position of at least one monosaccharide unit of the alkyl glycoside.
Clause 29. The method of any one of clauses 17-28, further comprising:
   dissolving the carbonate reaction product in an aqueous fluid.
Clause 30. The method of clause 29, wherein the aqueous fluid comprises at least one salt.

To facilitate a better understanding of the disclosure herein, the following examples of various representative embodiments are given. In no way should the following examples be read to limit, or to define, the scope of the invention.

### EXAMPLES

Reaction of an Alkyl Polyglycoside with CO₂ in the Presence of DBU. A commercial alkyl polyglycoside (APG1214, Shanghai Fine Chemical Co., Ltd., 2-3 glucose units and a C₁₂-C₁₄ alkyl group) was freeze dried to remove residual water prior to use. In a glass reaction vessel, 5 grams of the alkyl polyglycoside was dissolved in 100 mL dimethylsulfoxide, and a molar excess (3.1-4.3 g) of 1,8-diazabicyclo[5.4.0]undec-7-ene (DBU) was added. The reaction mixture was sealed and heated at a temperature of 50-60° C. while stirring. Carbon dioxide was then introduced to the sealed reaction vessel until a pressure of 3-5 bar was reached. The reaction was continued under heating conditions for several hours, during which time a white solid formed. The white solid was then isolated by centrifugation. The product was redissolved in deionized water, and the product was isolated by freeze drying.

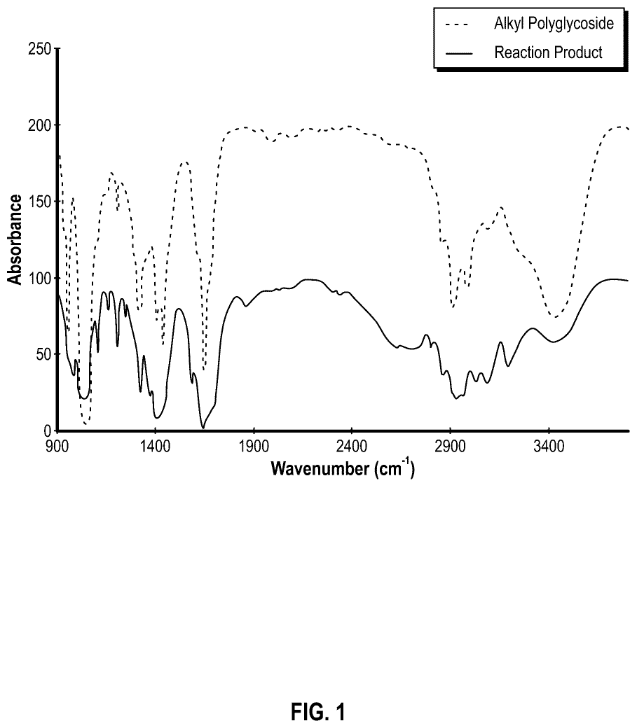

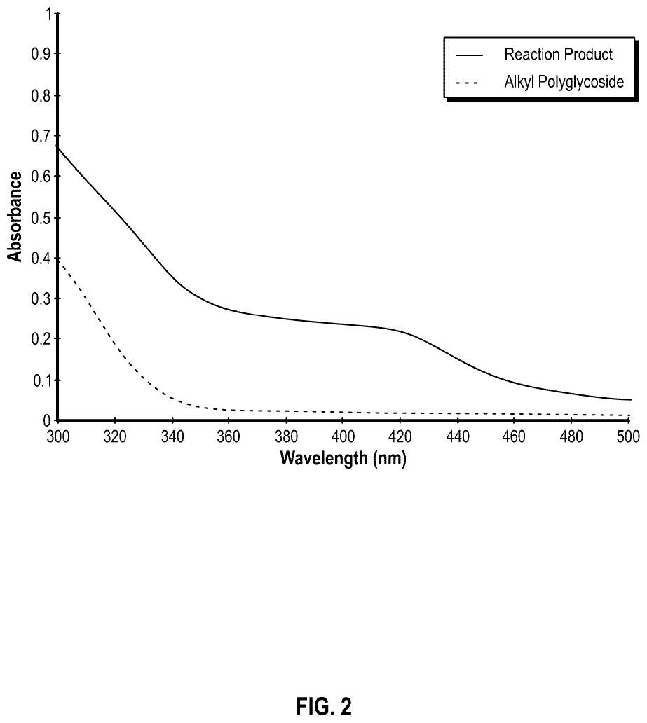

Spectroscopic Characterization. The carbonate salt reaction product was characterized by infrared and UV-VIS spectroscopic techniques. FIG. 1 is a plot of comparative FTIR spectra of the alkyl polyglycoside and the alkyl polyglycoside carbonate salt reaction product. Compared to the alkyl polyglycoside starting material, the reaction product contained a peak near 1600 cm⁻¹ that was broadened and contained new fine structure. The new peaks are characteristic of carbonate group introduction. Peaks related to adsorbed CO₂ were also evident near 2600 cm⁻¹. FIG. 2 is a plot of comparative UV-VIS spectra of the alkyl polyglycoside and the alkyl polyglycoside carbonate salt reaction product. As shown, the UV-VIS absorbance increased following formation of the carbonate salt reaction product. The increased UV-VIS absorbance is believed to result from the DBU cation.

Salt Tolerance. The carbonate salt reaction product was dissolved in high-salinity water having a total dissolved solids content of 57,670 ppm (18,300 ppm Na⁺, 650 ppm Ca²⁺, 2,110 ppm Mg²⁺, 4,290 ppm SO₄²⁻, 32,200 ppm Cl⁻, 120 ppm HCO₃) at a concentration of 0.2 wt. %. The solution remained transparent both at 25° C. and 90° C., indicating that precipitation did not occur in the presence of the high salt concentration.

Emulsification Performance. The foregoing aqueous solution of the carbonate salt reaction product in high-salinity water was combined with crude oil in a 1:1 volume ratio and emulsified therewith by shaking the mixture by hand. Oil-in-water emulsions readily formed upon agitation, and the emulsions separated into discrete oil and water phases after standing for 30 minutes or less. Emulsions formed only from the high-salinity water and crude oil, in contrast, did not undergo de-emulsification within the same timeframe.

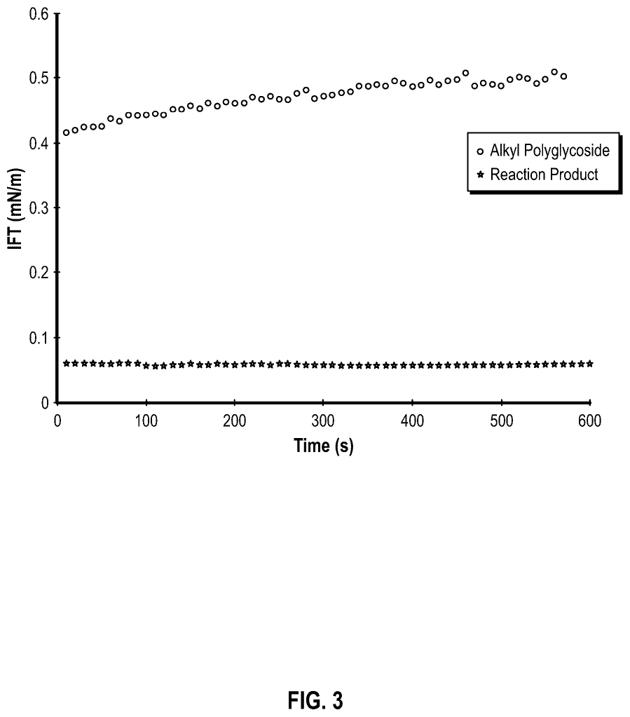

Interfacial Tension. Interfacial tension testing was performed on the foregoing high-salinity aqueous solutions using a spinning drop tensiometer (SVT 20, Dataphysics) at a rotation rate of 5000 rpm. FIG. 3 is a plot of interfacial tension as a function of time for the alkyl glycoside and the alkyl glycoside carbonate salt reaction product. As shown, the alkyl polyglycoside carbonate salt reaction product had approximately a 10-fold lower interfacial tension than did the parent alkyl polyglycoside. In addition, the interfacial tension of the carbonate salt reaction product remained relatively constant with time, unlike the parent alkyl polyglycoside, which displayed increasing interfacial tension with time.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

While the present disclosure has been described with respect to a limited number of embodiments, those skilled in the art, having benefit of this disclosure, will appreciate that other embodiments may be devised which do not depart from the scope of the disclosure as described herein. Accordingly, the scope of the disclosure should be limited only by the attached claims.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by one or more embodiments described herein. At the very least, and not as an attempt to limit the application of the doctrine of equivalents to the scope of the claim, each numerical parameter should at least be construed in light of the number of reported significant digits and by applying ordinary rounding techniques.

What is claimed is the following:

1. A composition comprising:
an organic carbonate salt comprising an anionic portion that is a carbonate reaction product of an alkyl glycoside.

2. The composition of claim 1, wherein the alkyl glycoside is an alkyl polyglycoside.

3. The composition of claim 2, wherein the alkyl polyglycoside comprises at least two monosaccharide units, one or more of the at least two monosaccharide units are functionalized with an organic carbonate group in salt form, and a terminal monosaccharide unit of the at least two monosaccharide units is alkylated.

4. The composition of claim 3, wherein the at least two monosaccharide units are linked by α-1,4-glycosidic bonds.

5. The composition of claim 1, wherein the organic carbonate salt comprises a cation portion comprising a non-nucleophilic base in protonated form.

6. The composition of claim 5, wherein the non-nucleophilic base has a pKₐ of about 10 or greater.

7. The composition of claim 5, wherein the non-nucleophilic base comprises an amidine.

8. The composition of claim 7, wherein the cation portion comprises 1,8-diazabicyclo[5.4.0]undec-7-ene (DBU) in protonated form.

9. The composition of claim 1, wherein the alkyl glycoside comprises a terminal alkyl group comprising about 4 to about 24 carbon atoms.

10. The composition of claim 1, wherein the carbonate reaction product comprises an organic carbonate group in salt form at a primary alcohol position of at least one monosaccharide unit of the alkyl glycoside.

11. The composition of claim 1, further comprising:
an aqueous fluid in which the organic carbonate salt is dissolved.

12. A method comprising:
providing the composition of claim 11; and
introducing the composition into a subterranean formation.

13. The method of claim 12, wherein the composition is introduced into the subterranean formation in conjunction with an enhanced hydrocarbon recovery process.

14. The method of claim 12, wherein the composition contacts a salt-containing fluid in the subterranean formation, the aqueous fluid comprises at least one salt, or any combination thereof.

15. The method of claim 12, further comprising:
contacting the composition with an oleaginous substance in the subterranean formation; and
emulsifying the oleaginous substance in the aqueous fluid in the presence of the organic carbonate salt.

16. The method of claim 12, wherein the composition is emulsified with an oleaginous substance when introduced into the subterranean formation.

17. A method comprising:
contacting an alkyl glycoside with an elevated pressure of carbon dioxide in the presence of a non-nucleophilic base; and
obtaining an organic carbonate salt comprising a cationic portion comprising the non-nucleophilic base in protonated form and an anionic portion that is a carbonate reaction product of the alkyl glycoside.

18. The method of claim 17, wherein contacting takes place in an aprotic organic solvent under heating.

19. The method of claim 18, wherein heating takes place at a temperature up to about 100° C.

20. The method of claim 17, wherein the elevated pressure is about 3 atm to about 5 atm.

21. The method of claim 17, wherein the alkyl glycoside is an alkyl polyglycoside.

22. The method of claim 21, wherein the alkyl polyglycoside comprises at least two monosaccharide units, one or more of the at least two monosaccharide units are functionalized with an organic carbonate group in salt form, and a terminal monosaccharide unit of the at least two monosaccharide units is alkylated.

23. The method of claim 22, wherein the at least two monosaccharide units are linked by α-1,4-glycosidic bonds.

24. The method of claim 17, wherein the non-nucleophilic base cation comprises an amidine.

25. The method of claim 24, wherein the cation portion comprises 1,8-diazabicyclo[5.4.0]undec-7-ene (DBU) in protonated form.

26. The method of claim 17, wherein the alkyl glycoside comprises a terminal alkyl group comprising about 4 to about 24 carbon atoms.
